(19) United States
(12) Patent Application Publication
(10) Pub. No.: US 2025/0186021 A1
(43) Pub. Date: Jun. 12, 2025

(54) METHOD AND SYSTEM FOR OBTAINING AN ULTRASOUND VOLUME FROM BI-PLANE ULTRASOUND SCANNING

(71) Applicant: GE Precision Healthcare LLC, Waukesha, WI (US)

(72) Inventors: Doron Shaked, Tivon (IL); Alexander Sokulin, Kiryat Tivon (IL); Radhika Madhavan, Latham, NY (US); Alexandra Gilinsky, Haifa (IL); Hannah Ornstein, Haifa (IL)

(21) Appl. No.: 18/530,515

(22) Filed: Dec. 6, 2023

(51) Int. Cl. A61B 8/14; A61B 8/08

(52) U.S. Cl. CPC A61B 8/145; A61B 8/483; A61B 8/5246

(57) ABSTRACT

Systems and methods for acquiring an ultrasound volume comprising acquiring sequential bi-plane ultrasound images in a first direction using an ultrasound probe of an ultrasound system, the sequential bi-plane ultrasound images comprising first ultrasound image planes or first ultrasound image volumes along a first plane and second ultrasound image planes or second ultrasound image volumes along a second plane, the first plane corresponding to the first direction and the second plane corresponding to a second direction, calculating one or more first displacements in the first direction of the sequential bi-plane ultrasound images during the acquisition, positioning the second ultrasound image planes or the second ultrasound image volumes sequentially, wherein the second ultrasound image planes or the second ultrasound image volumes are positioned according to the one or more first displacements, and generating the ultrasound volume by combining the second ultrasound image planes or the second ultrasound image volumes.

### FIELD

Certain embodiments relate to ultrasound imaging. More specifically, certain embodiments relate to a method and system for obtaining an ultrasound volume from bi-plane ultrasound images.

### BACKGROUND

Ultrasound imaging is a medical imaging technique for imaging anatomical structures, such as organs and soft tissues, in a human body. Ultrasound imaging uses real time, non-invasive high frequency sound waves to produce two-dimensional (2D) and three-dimensional (3D) ultrasound images.

Ultrasound imaging is a powerful tool for visualization. Ultrasound images are acquired by an ultrasound probe that may be used to scan anatomical structures to produce ultrasound images. However, current methods and ultrasound systems for acquiring a 3D volume require expensive equipment (e.g., 3D ultrasound probes), produce low quality ultrasound volumes, and/or are not able to produce volumes for large anatomies.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for obtaining an ultrasound volume from bi-plane ultrasound scanning, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to obtain an ultrasound volume from bi-plane ultrasound scanning, in accordance with various embodiments.

FIG. 2 is an example illustration of bi-plane ultrasound images with displacements along a first direction of movement that may be acquired to form an ultrasound volume, in accordance with various embodiments.

FIG. 3 is an example illustration of bi-plane ultrasound volumes with displacements along a first direction of movement that may be acquired to form an ultrasound volume, in accordance with various embodiments.

FIG. 4A is a graphical illustration of placement of second plane ultrasound images using displacements to form an ultrasound volume, in accordance with various embodiments.

FIG. 4B is a graphical illustration of combining the second plane ultrasound images to form an ultrasound volume, in accordance with various embodiments.

FIG. 5 is an example illustration of bi-plane ultrasound images along a first direction of movement and a second direction of movement that may be acquired to form an ultrasound volume, in accordance with various embodiments.

FIG. 6 is an example illustration of bi-plane ultrasound volumes along a first and second direction of movement that may be acquired to form an ultrasound volume, in accordance with various embodiments.

FIG. 7 is an example illustration of bi-plane ultrasound images with one or more rotations that may be acquired to form an ultrasound volume, in accordance with various embodiments.

FIG. 8 is an example illustration of geometric ultrasound volumes that may be acquired to form an ultrasound volume, in accordance with various embodiments.

FIG. 9 is an example artificial intelligence model that may be used to generate probe motion parameters in order to form an ultrasound volume from biplane ultrasound images, in accordance with various embodiments.

FIG. 10 is a flow chart illustrating exemplary steps 1102-1116 that may be utilized for obtaining an ultrasound volume from bi-plane ultrasound images, in accordance with various embodiments.

### DETAILED DESCRIPTION

Certain embodiments may be found in a method and system for obtaining an ultrasound volume from bi-plane ultrasound images. Aspects of the present disclosure have the technical effect of providing a full 3D volume of an ultrasound scan using a 2D probe. Various embodiments have the technical effect of rendering a 3D volume using 2D bi-plane ultrasound images. Certain embodiments have the technical effect of rendering a 3D volume by calculating displacements of bi-plane ultrasound images. Various embodiments have the technical effect of rendering a 3D volume of a large anatomical structure with a linear scan. Various embodiments have the technical effect of presenting an ultrasound operator with a rendering of a 3D volume that may be manipulated by the operator to present a particular view or to visualize an anatomical structure, although the view or visualization was not directly scanned by the ultrasound probe.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random access memory, hard disk, or the like) or multiple pieces of hardware. Similarly, the programs may be stand-alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized, and that structural, logical, and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising”, “including”, or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode, which can be one-dimensional (1D), two-dimensional (2D), three-dimensional (3D), or four-dimensional (4D), and comprising Brightness mode (B-mode), Motion mode (M-mode), Color Motion mode (CM-mode), Color Flow mode (CF-mode), Pulsed Wave (PW) Doppler, Continuous Wave (CW) Doppler, Contrast Enhanced Ultrasound (CEUS), and/or sub-modes of B-mode and/or CF-mode such as Harmonic Imaging, Shear Wave Elasticity Imaging (SWEI), Strain Elastography, Tissue Velocity Imaging (TVI), Power Doppler Imaging (PDI), B-flow, Micro Vascular Imaging (MVI), Ultrasound-Guided Attenuation Parameter (UGAP), and the like. The term, “ultrasound image,” as used herein, is used to refer to ultrasound image and/or ultrasound image volumes, such as a bi-plane image, a single 2D image, a rendering of a volume (3D/4D), 2D bi-plane image slices extracted from a volume (3D/4D), and/or any suitable ultrasound images.

Furthermore, the term processor or processing unit, as used herein, refers to any type of processing unit that can carry out the required calculations needed for the various embodiments, such as single or multi-core: CPU, Accelerated Processing Unit (APU), Graphic Processing Unit (GPU), Digital Signal Processor (DSP), Field-Programmable Gate Array (FPGA), Application-Specific Integrated Circuit (ASIC), or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. In addition, forming of images may be performed using channel combinations that may originate from more than one transmit event (e.g., synthetic aperture techniques).

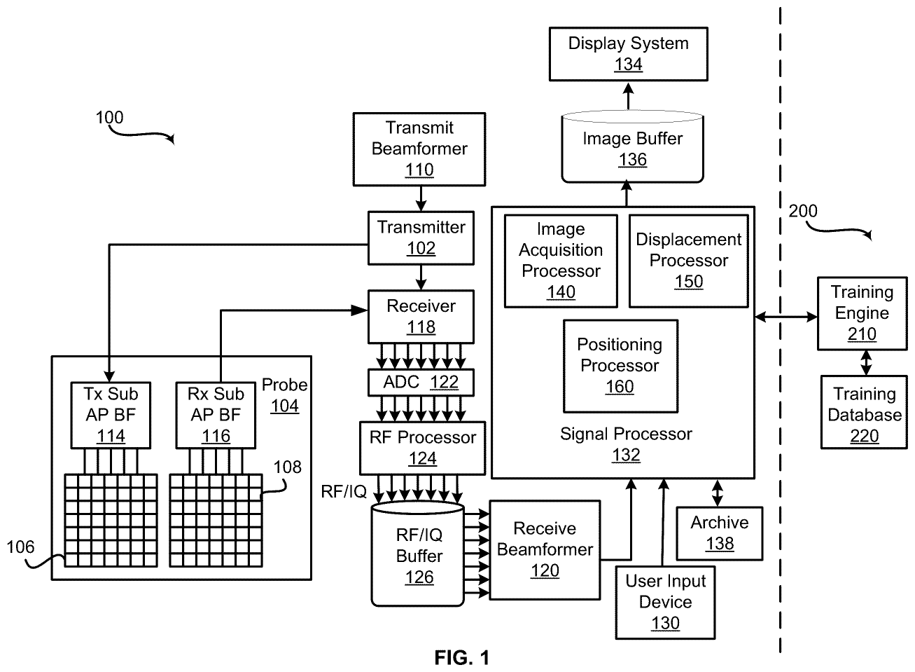

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to obtain an ultrasound volume from bi-plane ultrasound scanning. Referring to FIG. 1, there is shown an ultrasound system 100 and a training system 200. The ultrasound system 100 comprises a transmitter 102, an ultrasound probe 104, a transmit beamformer 110, a receiver 118, a receive beamformer 120, analog-to-digital (A/D) converters 122, a radio frequency (RF) processor 124, a RF quadrature (RF/IQ) buffer 126, a user input device 130, a signal processor 132, an image buffer 136, a display system 134, and an archive 138.

The transmitter 102 may comprise suitable logic, circuitry, interfaces, and/or code that may be operable to drive an ultrasound probe 104. The ultrasound probe 104 may comprise a two-dimensional (2D) array of piezoelectric elements. In various embodiments, the ultrasound probe 104 may be a matrix array transducer or any suitable transducer operable to acquire 2D and/or 3D ultrasound image datasets. The ultrasound probe 104 may comprise a group of transmit transducer elements 106 and a group of receive transducer elements 108, that normally constitute the same elements. In certain embodiment, the ultrasound probe 104 may be operable to acquire ultrasound image data covering at least a substantial portion of an anatomy, such as an abdomen, a heart, a fetus, a lung, a blood vessel, or any suitable anatomical structure(s).

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable logic, circuitry, interfaces and/or code that may be operable to receive the signals from the receive sub-aperture beamformer 116. The analog signals may be communicated to one or more of the plurality of A/D converters 122.

The plurality of A/D converters 122 may comprise suitable logic, circuitry, interfaces, and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces, and/or code that may be operable to demodulate the digital signals output by the plurality of A/D converters 122. In accordance with an embodiment, the RF processor 124 may comprise a complex demodulator (not shown) that is operable to demodulate the digital signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces, and/or code that may be operable to provide temporary storage of the RF or I/Q signal data, which is generated by the RF processor 124.

The receive beamformer 120 may comprise suitable logic, circuitry, interfaces and/or code that may be operable to perform digital beamforming processing to, for example, sum the delayed channel signals received from RF processor 124 via the RF/IQ buffer 126 and output a beam summed signal. The resulting processed information may be the beam summed signal that is output from the receive beamformer 120 and communicated to the signal processor 132. In accordance with some embodiments, the receiver 118, the plurality of A/D converters 122, the RF processor 124, and the beamformer 120 may be integrated into a single beamformer, which may be digital. In various embodiments, the ultrasound system 100 comprises a plurality of receive beamformers 120.

The user input device 130 may be utilized to input patient data, scan parameters, settings, select protocols and/or templates, select displacement parameters to acquire displacements in one more directions and/or rotational displacements, manipulate the acquired 3D volume, and the like. In an exemplary embodiment, the user input device 130 may be operable to configure, manage, and/or control operation of one or more components and/or modules in the ultrasound system 100. In this regard, the user input device 130 may be operable to configure, manage, and/or control operation of the transmitter 102, the ultrasound probe 104, the transmit beamformer 110, the receiver 118, the receive beamformer 120, the RF processor 124, the RF/IQ buffer 126, the user input device 130, the signal processor 132, the image buffer 136, the display system 134, and/or the archive 138. The user input device 130 may include button(s), rotary encoder(s), a touchscreen, motion tracking, voice recognition, a mousing device, keyboard, camera, and/or any other device capable of receiving a user directive. In certain embodiments, one or more of the user input devices 130 may be integrated into other components, such as the display system 134 or the ultrasound probe 104, for example. As an example, user input device 130 may include a touchscreen display.

The signal processor 132 may comprise suitable logic, circuitry, interfaces and/or code that may be operable to process ultrasound scan data (i.e., summed IQ signal) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 134 and/or may be stored at the archive 138. The archive 138 may be a local archive, a Picture Archiving and Communication System (PACS), a remote archive, or any suitable device for storing images and related information.

The signal processor 132 may be one or more central processing units, microprocessors, microcontrollers, and/or the like. The signal processor 132 may be an integrated component, or may be distributed across various locations, for example. In an exemplary embodiment, the signal processor 132 may comprise an image acquisition processor 140, a displacement processor 150, and a positioning processor 160. The signal processor 132 may be capable of receiving input information from a user input device 130 and/or archive 138, generating an output displayable by a display system 134, and manipulating the output in response to input information from a user input device 130, among other things. The signal processor 132, image acquisition processor 140, displacement processor 150, and/or positioning processor 160 may be capable of executing any of the method(s) and/or set(s) of instructions discussed herein in accordance with the various embodiments, for example.

The ultrasound system 100 may be operable to continuously acquire ultrasound scan data at a frame rate that is suitable for the imaging situation in question. Typical frame rates range from 20-120 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data. The frames of ultrasound scan data are stored in a manner to facilitate retrieval thereof according to its order or time of acquisition. The image buffer 136 may be embodied as any known data storage medium.

The signal processor 132 may include an image acquisition processor 140 that comprises suitable logic, circuitry, interfaces, and/or code that may be operable to acquire ultrasound images of anatomical structures such as cardiac structures, gastroenterological structures, urological structures, reproductive structures, cardiac structures, pulmonary structures, and/or any suitable anatomical structures. The ultrasound images may be ultrasound images and/or ultrasound image volumes, such as a bi-plane image, a single 2D image, a rendering of a volume (3D/4D), 2D bi-plane image slices extracted from a volume (3D/4D), and/or any suitable ultrasound images.

In an exemplary embodiment, the image acquisition processor 140 may acquire a series of ultrasound images using an ultrasound probe 104 that is moved across an anatomical structure. The image acquisition processor 140 may capture the series of ultrasound images along a first direction of movement corresponding to a first plane. Additionally and/or alternatively, the image acquisition processor 140 may capture a series of ultrasound images along a second direction of movement corresponding to the second plane, a series of ultrasound images along additional different directions of movement, and/or a series of ultrasound images along one or more axes of rotation.

The ultrasound images may be bi-plane ultrasound images including an ultrasound image in the first plane and an ultrasound image in the second plane. The first and second planes may be at an angle from each other. In some embodiments, the two planes are orthogonal to each other. In some examples, the first plane is an elevation plane and the second plane is an azimuth plane. As the ultrasound probe 104 scans linearly along the first plane, in the first direction, the ultrasound probe captures a series of bi-plane ultrasound images. When capturing ultrasound images as ultrasound image volumes, the ultrasound probe may capture 2D bi-plane images, image slices, or ultrasound image volumes. In some examples, a subset of image 2D bi-plane images or image slices may be combined to form the ultrasound image volume(s). Ultrasound image volumes may then be obtained sequentially as the ultrasound probe 104 is moved across the anatomical structures. The bi-plane ultrasound images and/or ultrasound image volumes may be stored in an archive, and/or may be provided to the displacement processor 150 and/or the positioning processor 150.

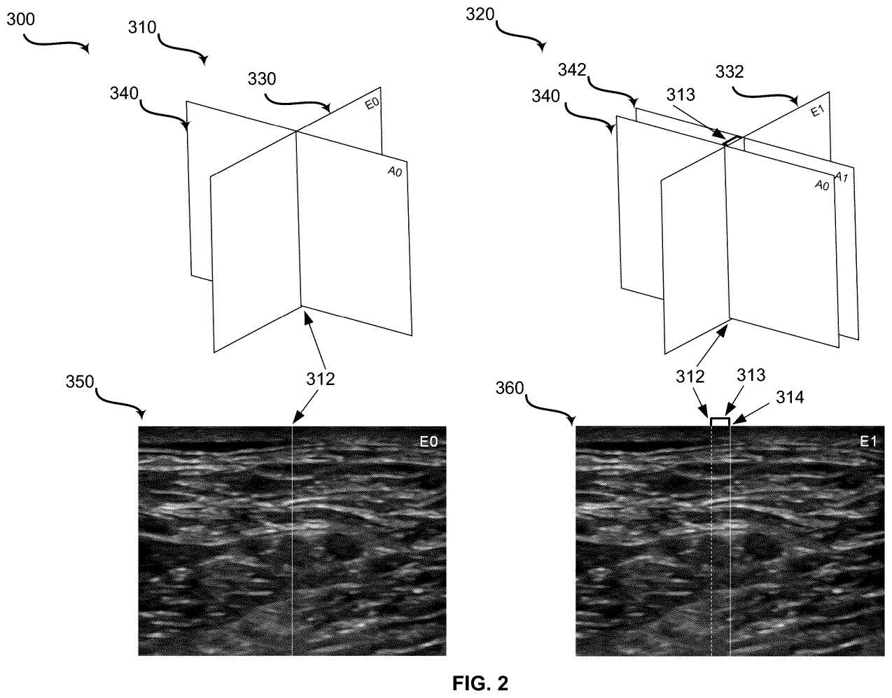

FIG. 2 provides an example illustration 300 of bi-plane ultrasound images with displacements along a first direction of movement that may be acquired to form an ultrasound volume. The illustration 300 includes an exemplary diagram 310 of intersecting ultrasound images, an exemplary diagram 320 of displacements of the ultrasound images, a first ultrasound image 350, and a second ultrasound image 360. The exemplary diagram 310 includes first plane representation 330 and second plane representation 340, which may be on an elevation plane and an azimuth plane, respectively. The first plane representation 330 and the second plane representation 340 intersect at center line 312. In some examples, the first plane representation 330 and the second plane representation 340 are at an angle relative to each other. In some examples, the first plane ultrasound representation 330 is orthogonal to the second plane representation 340 (e.g., the first plane representation 330 is at a 90 degree angle to the second plane representation 340). Exemplary diagram 320 includes a first plane representation 332, a second plane representation 340, and a second plane representation 342. In some examples, the first plane is an elevation plane and the second plane is an azimuth plane.

As an ultrasound probe 104 is moved approximately linearly across an anatomical structure, the image acquisition processor 140 captures ultrasound images while traveling along the first direction of movement. For example, ultrasound image 350 may be a first 2D bi-plane ultrasound image captured along the elevation plane. Ultrasound image 360 may be a second 2D bi-plane ultrasound image along the elevation plane. First plane representation 330 may be a representation of ultrasound image 350 and first plane representation 332 may be a representation of ultrasound image 360. Ultrasound images 350, 360 depict center line 312. Ultrasound image 360 additionally depicts displacement line 314, which represents an intersection of the first plane representation 332 and second plane representation 342. Because the ultrasound probe 104 is traveling linearly along the first plane (e.g., elevation plane), first plane representation 330 and first plane representation 332 are on the same plane (e.g., first plane, elevation plane, etc.), and a first displacement 313 may be calculated from a movement of the intersection of the first plane and the second plane (e.g., center line 312) on the ultrasound image 350, 360 to displacement line 314 on ultrasound image 360. In some examples, the first displacement 313 between the displacement line 314 and the center line 312 may be calculated by the displacement processor 150.

Returning to FIG. 1, the signal processor 132 may include a displacement processor 150 that comprises suitable logic, circuitry, interfaces, and/or code that may be operable to calculate displacement information for a series of ultrasound images acquired by the image acquisition processor 140. For example, the displacement processor 150 may be configured to receive from the image acquisition processor 140, or retrieve from the archive 138 and/or any suitable data storage medium, the series of ultrasound images in order to calculate displacements between each of the acquired ultrasound images.

A displacement may be calculated from a center line of the first plane ultrasound image to one or more displacement lines in subsequent ultrasound images of the series of ultrasound images by a displacement processor 150. For example, the series of ultrasound images may be bi-plane ultrasound images which include first plane ultrasound images and second plane ultrasound images. The center line may be an intersection of the first plane and the second plane on a first plane ultrasound image. After movement in a linear direction along the first plane, the displacement from a new intersection of the first plane and the second plane to the center line captured on a subsequent first plane ultrasound image may be calculated. In some examples, the displacement from the center line may be used as the displacement for the remaining series of ultrasound images (e.g., the displacement between each of the images in the series of ultrasound images). In some other examples, a displacement may be calculated from the intersection of the first plane and second plane of each of the subsequent first plane ultrasound images to the intersection of each of the previous first plane ultrasound images. The displacements calculated by the displacement processor 150 may be stored and/or provided to the positioning processor 160.

In some examples, the displacement processor 150 may calculate displacements between a series of sequential ultrasound images obtained by the ultrasound probe 104 in a first direction, along a first plane. Additionally and/or alternatively, the displacement processor 150 may calculate displacements in a second direction along a second plane that is at an angle relative to the first plane. The displacement processor 150 may calculate displacements for additional planes along additional directions. Additionally and/or alternatively, the displacement processor 150 may calculate rotational displacements along one or more axes for the series of ultrasound images. The displacement processor 150 may calculate displacements along one or more planes and/or rotational displacements individually, simultaneously, and/or in a sequential order. In some examples, the displacement processor 150 may assume a null or zero displacement or zero rotation while calculating planar displacements and/or rotational displacements. In some examples, the displacement processor 150 may calculate displacements along one or more planes and/or rotational displacements using normalized cross-correlation between each of the series of ultrasound images.

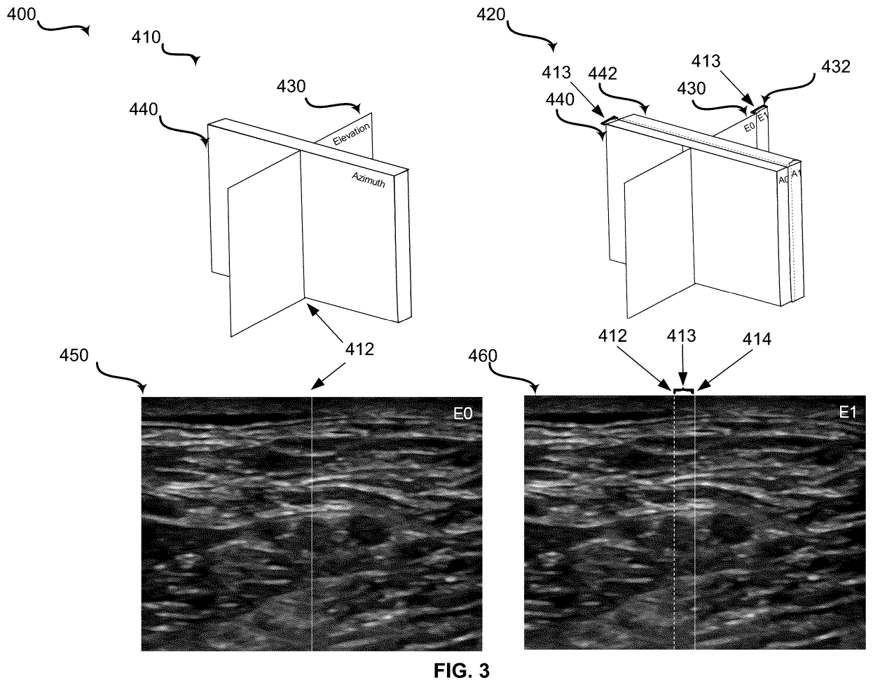

FIG. 3 is an example illustration 400 of bi-plane ultrasound volumes with displacements along a first direction of movement that may be acquired to form an ultrasound volume. The illustration 400 includes an exemplary diagram 410 of a first plane 430 intersecting a second plane representation volume 440, an exemplary diagram 420 of displacements, a first ultrasound image 450, and a second ultrasound image 460.

The exemplary diagram 410 includes a first plane representation 430 (e.g. along an elevation plane) and a representation of a second plane representation volume 440 (e.g. along an azimuth plane). The first plane representation 430 and the second plane representation volume 440 intersect at center line 412. In some examples, the first plane representation 430 and the second plane representation volume 440 are at an angle relative to each other. In some examples, the first plane representation 430 is orthogonal to the second plane representation volume 440 (e.g., the first plane ultrasound image is at a 90 degree angle to the second plane representation volume 440). Exemplary diagram 420 includes first plane representation 432, second plane representation volume 440, and second plane representation volume 442. In some examples, second plane representation volume 440 and second plane representation volume 442 overlap.

As an ultrasound probe 104 is moved approximately linearly across an anatomical structure, the image acquisition processor 140 captures ultrasound images while traveling along the first direction of movement. For example, ultrasound image 450 may be a first 2D bi-plane ultrasound image captured along the elevation plane. Ultrasound image 460 may be a second 2D bi-plane ultrasound image along the elevation plane. First plane representation 430 may be a representation of ultrasound image 450 and first plane representation 432 may be a representation of ultrasound image 460. Ultrasound images 450, 460 depict center line 412. Ultrasound image 460 additionally depicts displacement line 414, which represents an intersection of the first plane representation 432 and second plane representation volume 442. A displacement 413 may be calculated between second plane representation volume 440 and second plane representation volume 442. In some examples, the displacement 413 may be calculated between the displacement line 414 and the center line 412 by the displacement processor 150. A second displacement may be calculated from the second plane representation volume to a subsequent ultrasound image volume. Additional displacements may be calculated for a series of image volumes along the same plane. The displacements and/or additional displacements calculated by the displacement processor 150 may be stored and/or provided to the positioning processor 160.

Referring back to FIG. 1, the signal processor 132 may include a positioning processor 160 that comprises suitable logic, circuitry, interfaces, and/or code that may be operable to cause a display system 134 to present an ultrasound image volume based on the displacements of the series of ultrasound images obtained by the ultrasound probe 104, in accordance with various embodiments. The displacements of the sequential ultrasound images may be received from the displacement processor 150 and/or retrieved from the archive 138.

In some examples, the displacements measured from the first plane ultrasound images of the series of bi-plane ultrasound images may be utilized by the positioning processor 160 to form ultrasound volumes by using the displacements to position second plane ultrasound images of the bi-plane ultrasound images according to the displacements in the first plane ultrasound images. For example, the positioning processor 160 may obtain the displacements along a first plane for 2D bi-plane ultrasound images, which include bi-plane ultrasound images along a first plane (e.g., first plane ultrasound image) and along a second plane (e.g., second plane ultrasound image), and use the displacements along the first plane to position the second plane ultrasound images sequentially according to the displacements calculated along the first plane. In some examples, the first plane is an elevation plane, and the second plane is an azimuth plane.

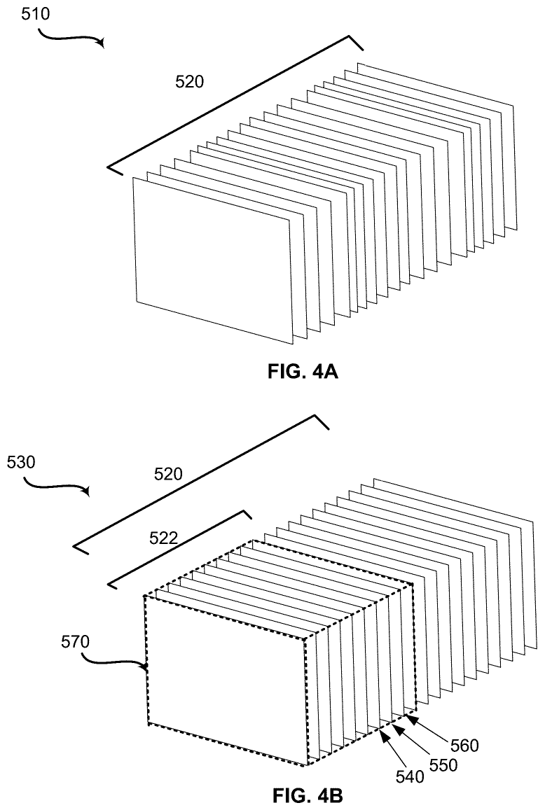

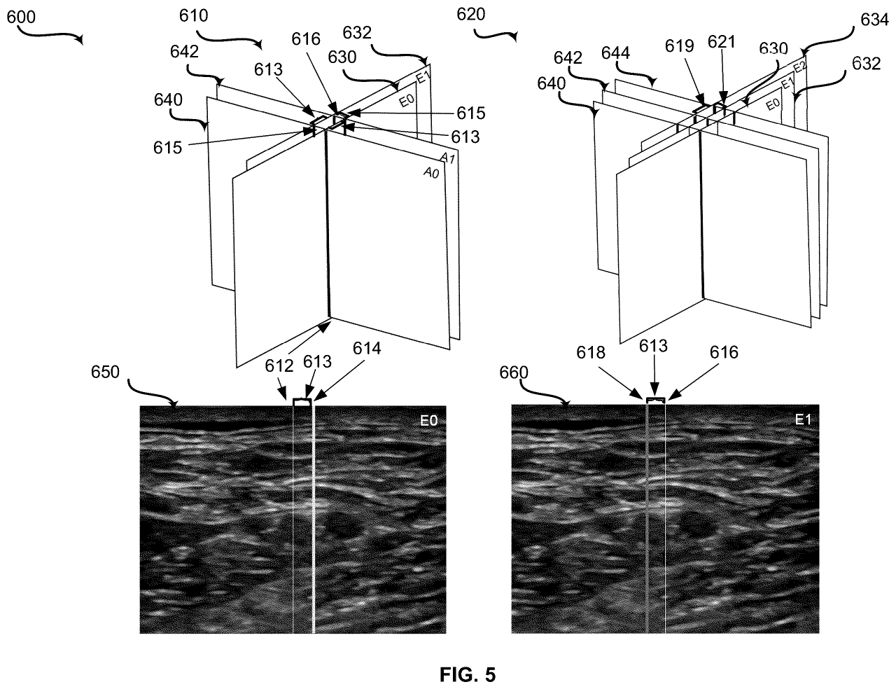

FIG. 4A provides a graphical illustration 510 of placement of the second plane ultrasound images 520 using displacements described above with regards to FIGS. 2 and 3 (and below with regards to FIGS. 5-9). FIG. 4B provides a graphical illustration 530 of combining the second plane ultrasound images 520 to form an ultrasound volume 570. Referring to FIG. 4A, the series of second plane ultrasound images 520 may be placed in sequential order using the displacements calculated along the first plane as described above. For example, the positioning processor 160 may obtain the displacements along a first plane for 2D bi-plane ultrasound images, which include bi-plane ultrasound images along a first plane and along a second plane, and use the displacements along the first plane to position the second plane ultrasound images sequentially using the displacements calculated along the first plane. The series of ultrasound images 520 may be images and/or image volumes.

Referring to FIG. 4B, graphical illustration 530 provides combining the second plane ultrasound images 520 to form an ultrasound volume 570 once the second plane ultrasound images 520 are positioned by the positioning processor. In some examples, the positioning processor 160 may form a 3D ultrasound volume by combining all of the second plane ultrasound images 520. In some other examples, the positioning processor 160 may form a 3D ultrasound volume by combining a subset 522 of the second plane ultrasound images 520. For example, second plane ultrasound image 540 may be combined with second plane ultrasound image 550 and second plane ultrasound image 560. Additionally and/or alternatively, second plane ultrasound images 540, 550, 560 may be combined with additional second plane ultrasound images in subset 522 to form an ultrasound volume 570. In some examples, such as when the ultrasound images 520 are image volumes, the ultrasound images 520 may overlap and combining the ultrasound images may account for any overlapping between the ultrasound images 520. In some other examples, such as when the ultrasound images are 2D images, the ultrasound images 520 may have a space or gap between the ultrasound images 520 and the ultrasound images 520 may be interpolated. In some examples, the ultrasound images 520 may be interpolated, for example, by calculating approximate values for the spaces or gaps in between the ultrasound images and using the calculated approximate values when forming the 3D ultrasound volume. In some examples, the positioning processor 160 may store the 3D ultrasound volume information in an archive 138 and/or may render the 3D ultrasound volume on the display 134.

FIG. 5 is an example illustration 600 of 2D bi-plane ultrasound images along a first direction of movement and a second direction of movement that may be acquired to form an ultrasound volume. The illustration 600 includes an exemplary diagram 610 of two intersecting ultrasound image representations, an exemplary diagram 620 of displacements, a first ultrasound image 650, and a second ultrasound image 660. The exemplary diagram 610 includes a first plane representation 630, a first plane representation 632, a second plane representation 640, and a second plane representation 642. In some examples, the first plane may be an elevation plane and the second plane may be an azimuth plane. The first plane representation 630 and the second plane representation 640 intersect at center line 612. In some examples, the first plane representation 630 and the second plane representation 640 are at an angle relative to each other. In some examples, the first plane representation 630 is orthogonal to the second plane representation 640 (e.g., the first plane representation 630 is at a 90 degree angle to the second plane representation 640). In some examples, the first plane is an elevation plane and the second plane is an azimuth plane. Exemplary diagram 620 includes similar elements as exemplary diagram 610, and also includes first plane representation 634 and second plane representation 644.

As an ultrasound probe 104 is moved across an anatomical structure, the image acquisition processor 140 captures ultrasound images while traveling along first and second directions of movement. For example, ultrasound images 650, 660 may be 2D bi-plane ultrasound images captured along the elevation plane. First plane representation 630 along the elevation plane may be a representation of ultrasound image 650 and first plane representation 632 may be a representation of ultrasound image 660 along the elevation plane. Ultrasound images 650 depicts center line 612, which represents an intersection of first plane representation 630 and second plane representation 640. Ultrasound image 650 additionally depicts first plane displacement 614 to the right of center line 612, which represents an intersection of the first plane representation 630 and second plane representation 642. Ultrasound image 660 depicts center line 616, which is an intersection of first plane representation 632 and second plane representation 642. Ultrasound image 660 additionally depicts displacement line 618 to the left of center line 616, which represents an intersection of the first plane representation 632 and second plane representation 640. In some examples, a displacement 613 of the first plane between the displacement line 614 and the center line 612 may be calculated by the displacement processor 150.

In some examples, a second displacement along the second plane may also be calculated. For example, a displacement 615 between first plane representation 630 and first plane representation 632 may be calculated along second plane representation 642 and along second plane representation 640. In some examples, additional displacements may be calculated between the series of ultrasound images obtained by the acquisition processor 140. For example, exemplary diagram 620 depicts additional displacements 619, 621 calculated for first plane representation 634 and second plane representation 644 representing additional ultrasound images from the series of ultrasound images acquired by the image acquisition processor 140. In some examples, the additional displacements 619, 621 may be measured at different areas of the ultrasound images and may be used to assess accuracy of the additional displacements 619, 621, and/or other displacement measurements, such as displacement 613 and/or displacement 615. The displacements and/or additional displacements calculated by the displacement processor 150 may be stored in an archive 138 and/or provided to the positioning processor 160.

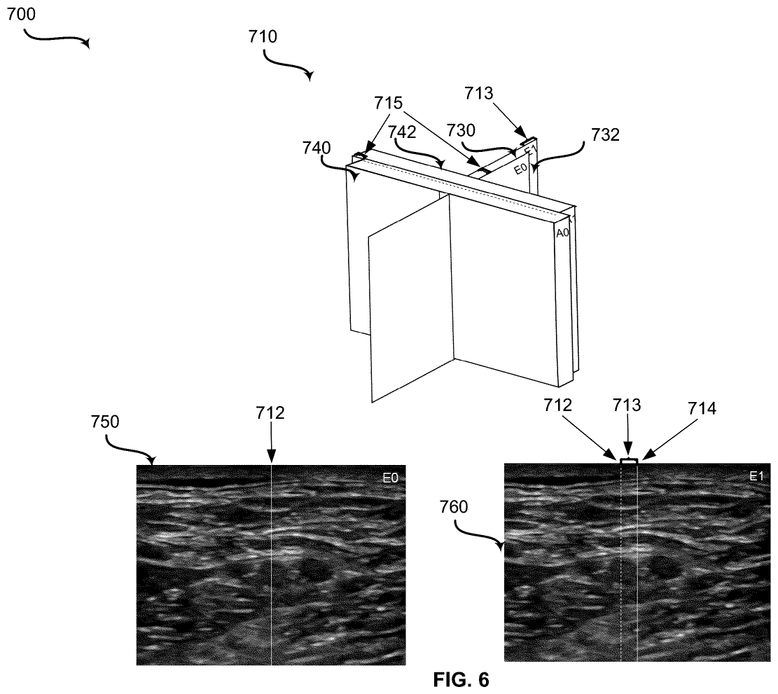

FIG. 6 is an example illustration 700 of bi-plane ultrasound volumes along a first and second direction of movement that may be acquired to form an ultrasound volume. The illustration 700 includes an exemplary diagram 710 of displacements of volumes in a first direction and a second direction, a first ultrasound image 750, and a second ultrasound image 760.

The exemplary diagram 710 includes a first plane representation 730, a first plane representation 732, a second plane representation volume 740 (e.g. along an azimuth plane), and a second plane representation volume 742. The first plane representation 730 and the second plane representation volume 740 intersect at center line 412. In some examples, the first plane representation 730 and the second plane representation volume 740 are at an angle relative to each other. In some examples, the first plane representation 730 is orthogonal to the second plane representation volume 740 (e.g., the first plane ultrasound image is at a 90 degree angle to the second plane representation volume 740). In some examples, second plane representation volume 740 and second plane representation volume 742 overlap.

As an ultrasound probe 104 is moved approximately linearly across an anatomical structure, the image acquisition processor 140 captures ultrasound images while traveling along the first direction of movement. For example, ultrasound image 750 may be a first 2D bi-plane ultrasound image captured along the elevation plane. Ultrasound image 760 may be a second 2D bi-plane ultrasound image along the elevation plane. First plane representation 730 may be a representation of ultrasound image 750 and first plane representation 732 may be a representation of ultrasound image 760. Ultrasound images 750, 760 depict center line 712. Ultrasound image 760 additionally depicts displacement line 714, which represents an intersection of the first plane representation 732 and second plane representation volume 742. A displacement 713 may be calculated between second plane representation volume 740 and second plane representation volume 742. In some examples, the displacement 713 may be calculated between the displacement line 714 and the center line 712 by the displacement processor 150. A second displacement may be calculated from the second plane representation volume to a subsequent ultrasound image volume along the first plane (e.g., elevation plane). Additional displacements may be calculated for a series of image volumes along the first plane.

In some examples, a displacement along the second plane may also be calculated. For example, a displacement 715 between first plane representation 730 and first plane representation 732 may be calculated along second plane representation volume 742 and along second plane representation volume 740. In some examples, additional displacements may be calculated between the series of ultrasound images obtained by the acquisition processor 140.

In some examples, the displacements 713, 715 may be measured at different locations on the ultrasound images and may be used to assess accuracy of the displacements 713, 715, and/or other displacement measurements. The displacements and/or additional displacements calculated by the displacement processor 150 may be stored in an archive 138 and/or provided to the positioning processor 160.

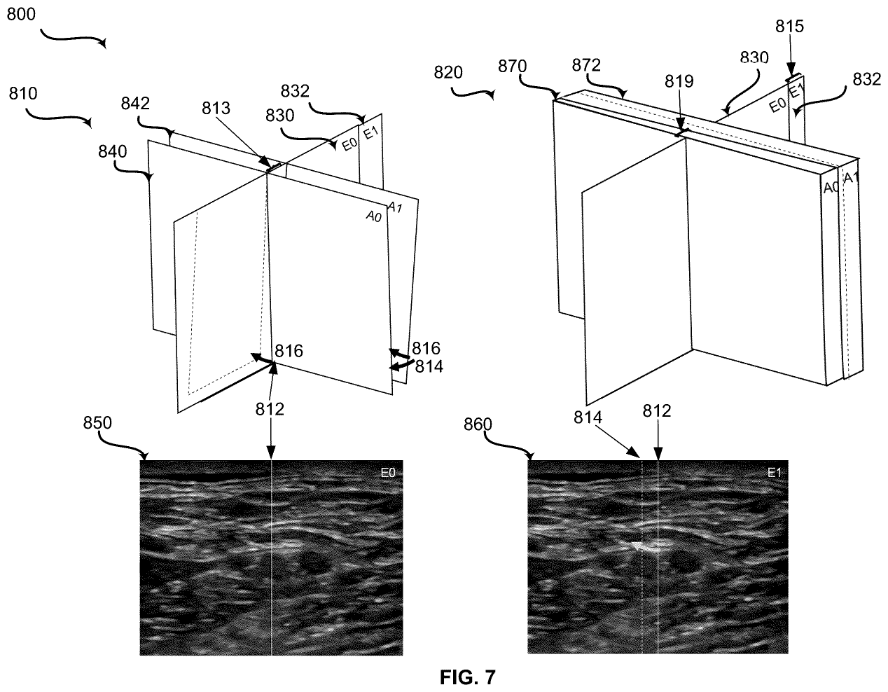

FIG. 7 is an example illustration 800 of bi-plane ultrasound images with one or more rotations that may be acquired to form an ultrasound volume. The illustration 800 includes an exemplary diagram 810 of ultrasound images with rotational displacements, an exemplary diagram 820 of ultrasound volumes with rotational displacements, a first ultrasound image 850, and a second ultrasound image 860. The exemplary diagram 810 includes a first plane representation 830, a first plane representation 832, a second plane representation 840, and a second plane representation 842. In some examples, the first plane may be an elevation plane and the second plane may be an azimuth plane. The first plane representation 830 and the second plane representation 840 intersect at center line 812. In some examples, the first plane representation 830 and the second plane representation 840 are at an angle relative to each other. In some examples, the first plane representation 830 is orthogonal to the second plane representation 840 (e.g., the first plane representation 830 is at a 90 degree angle to the second plane representation 840). In some examples, the first plane is an elevation plane and the second plane is an azimuth plane.

As an ultrasound probe 104 is moved across an anatomical structure, the image acquisition processor 140 captures ultrasound images while rotating with a rotation 814 and/or rotation 816. For example, ultrasound images 850, 860 may be 2D bi-plane ultrasound images captured along the elevation plane. First plane representation 830 along the elevation plane may be a representation of ultrasound image 850 and first plane representation 832 may be a representation of ultrasound image 860 along the elevation plane. Ultrasound images 850 depicts center line 812, which represents an intersection of first plane representation 830 and second plane representation 840. Ultrasound image 860 depicts center line 812 and first rotation 814 to the left of center line 812, which represents an intersection of the first plane representation 830 and second plane representation 842. A rotation 814 may be calculated by the displacement processor 150 which measures offset and/or rotation between first plane representation 830 and first plane representation 832.

In some examples, a rotation 816 may also be calculated by the displacement processor 150. For example, a rotation 816 of second plane representation 840 and second plane representation 842 may be calculated. In some examples, additional rotations may be calculated between series of ultrasound images obtained by the acquisition processor 140. In some examples, the additional rotations may be measured at different areas/locations of the ultrasound images and may be used to assess accuracy of the additional rotations and/or other displacement measurements. The rotations, additional rotations, and/or displacements calculated by the displacement processor 150 may be stored in an archive 138 and/or provided to the positioning processor 160.

Exemplary diagram 820 includes the first plane representation 830, the first plane representation 832, a second plane representation volume 870, and a second plane representation volume 872. In some examples, the first plane representation 830 and the second plane representation volume 870 are at an angle relative to each other. In some examples, the first plane representation 830 is orthogonal to the second plane representation volume 870 (e.g., the first plane representation 830 is at a 90 degree angle to the second plane representation volume 870). In some examples, second plane representation volume 870 and second plane representation volume 872 overlap.

First plane representation 830 may be a representation of ultrasound image 850 and first plane representation 832 may be a representation of ultrasound image 860. A rotation 819 may be calculated between second plane representation volume 870 and second plane representation volume 872. Additional rotations may be calculated from the second plane representation volume 872 to subsequent ultrasound image volumes about the first plane (e.g., elevation plane). Additional rotations may also be calculated for a series of ultrasound images about the second plane. In some examples, additional displacements may be calculated between the series of ultrasound images obtained by the acquisition processor 140.

In some examples, the rotations 814, 816, 819 may be measured at different locations on the ultrasound images and may be used to assess accuracy of the 814, 816, 819, and/or other displacement measurements. The rotations and/or additional rotations calculated by the displacement processor 150 may be stored in an archive 138 and/or provided to the positioning processor 160.

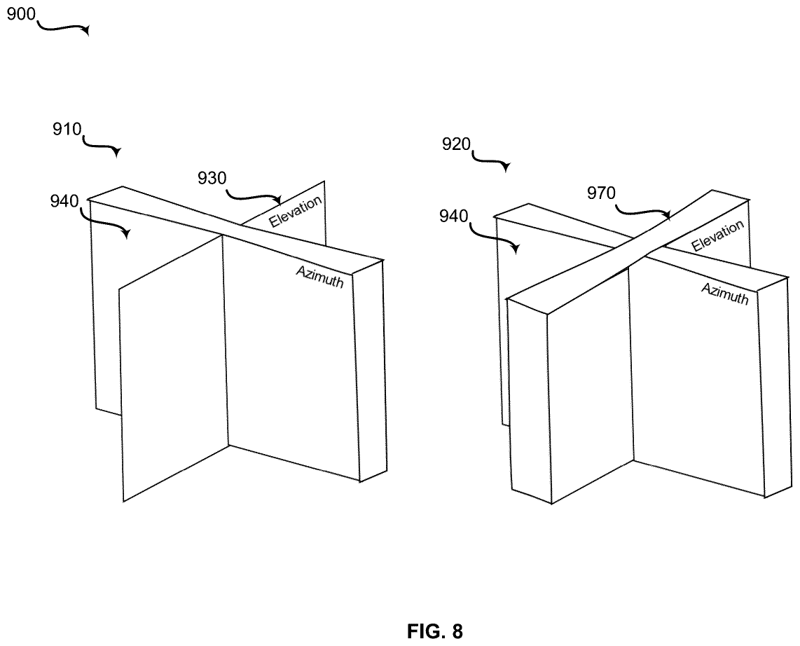

FIG. 8 is an example illustration 900 of geometric ultrasound volumes that may be acquired to form an ultrasound volume. The illustration 900 includes an exemplary diagram 910 of displacements using a first plane representation 930 intersecting a second plane representation volume 940, an exemplary diagram 920 of displacements using a first plane representation volume 970, and a second plane representation volume 940. The exemplary diagram 910 includes a first plane representation 930 (e.g. along an elevation plane) and a second plane representation volume 940 (e.g. along an azimuth plane). The first plane representation 930 and the second plane representation volume 440 intersect. In some examples, the first plane representation 930 and the second plane representation volume 940 are at an angle relative to each other. In some examples, the first plane representation 930 is orthogonal to the second plane representation volume 940 (e.g., the first plane ultrasound image is at a 90 degree angle to the second plane representation volume 940). Exemplary diagram 920 includes first plane representation volume 970 and second plane representation volume 940.

As an ultrasound probe 104 is moved approximately linearly across an anatomical structure, the image acquisition processor 140 captures ultrasound images while traveling along the first direction and/or second direction of movement. For example, first plane representation 930 and first plane representation volume 970 may be captured along the elevation plane. Second plane representation volume 940 may be captured on the azimuth plane. Because each of the first plane representation volume 970 and second plane representation volume 940 are geometric volumes, several additional planes may be captured while scanning the first plane representation volume and the second plane representation volume 940.

Displacements (not shown) may be calculated between second plane representation volume 940 and subsequent second plane representation volumes and/or between first plane representation volume 970 and subsequent first plane representation volumes. In some examples, the displacement(s) may be calculated from an intersection of the first plane representation 930 and the second plane representation volume 940 by the displacement processor 150. Additional displacements may be calculated for a series of geometric volumes along the same plane. The displacements and/or additional displacements calculated by the displacement processor 150 may be stored and/or provided to the positioning processor 160.

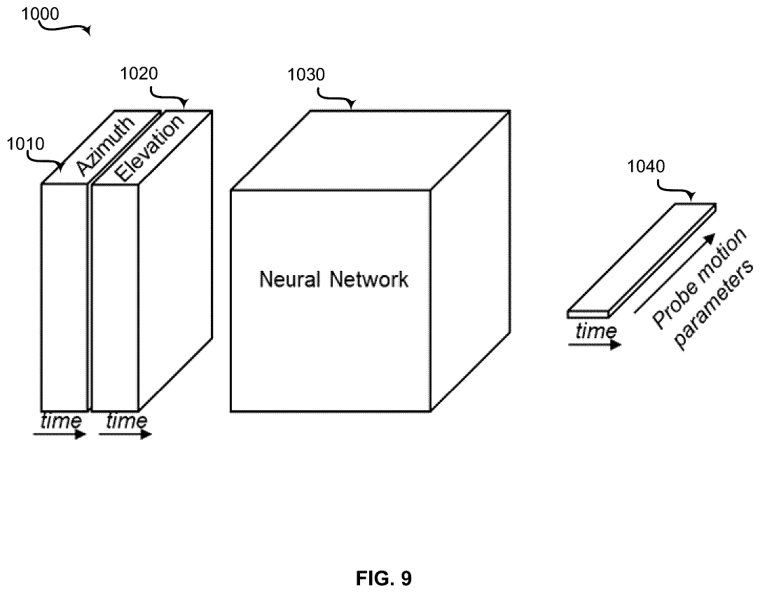

FIG. 9 is an example artificial intelligence model that may be used to generate probe motion parameters in order to form an ultrasound volume from ultrasound images. For example, motion of an ultrasound probe 104 may be analyzed in order to capture movement (e.g., along one or more plane, and/or one or more rotations) of an ultrasound probe 104 as the ultrasound probe captures ultrasound images. The motion of the ultrasound probe 104 may be used by a neural network to generate probe motion parameters 1040 and/or time data. In some examples, the ultrasound probe 104 motion parameters 1040 may include displacements of one or more planes and/or rotation for a series of bi-plane images. The probe motion parameters 1040 and/or time data may be stored in a training database, such as training database 220. In some examples, the positioning processor 160 may obtain probe motion parameters, displacement information, and/or rotation information in order to position a series of ultrasound images to construct 3D ultrasound volumes.

Referring back to FIG. 1, the displacement processor 150 may comprise suitable logic, circuitry, interfaces, and/or code that may be operable to analyze acquired ultrasound probe motion of an ultrasound operator to detect probe motion parameters over time in one or more planes (e.g. azimuth, elevation, etc.) and to store the probe motion parameters and/or provide the probe motion parameters to the positioning processor 160 in order to construct a 3D ultrasound volume of anatomical structures (e.g., cardiac structures, gastroenterological structures, urological structures, reproductive structures, cardiac structures, pulmonary structures, and/or any suitable anatomical structures) from obtained bi-plane ultrasound images. In this regard, the displacement processor 150 may include, for example, motion and/or image analysis algorithms, one or more deep neural networks (e.g., a convolutional neural network such as u-net) and/or may utilize any suitable form of motion and/or image analysis techniques, artificial intelligence, or machine learning processing functionality configured to detect motion parameters of an ultrasound probe gathering ultrasound images.

Additionally and/or alternatively, the motion and/or image analysis techniques, artificial intelligence, or machine learning processing functionality configured to detect and motion parameters of an ultrasound probe gathering ultrasound images may be provided by a different processor or distributed across multiple processors at the ultrasound system 100 and/or a remote processor communicatively coupled to the ultrasound system 100. For example, the motion and/or image analysis functionality may be provided as a deep neural network that may be made up of, for example, an input layer, an output layer, and one or more hidden layers in between the input and output layers. Each of the layers may be made up of a plurality of processing nodes that may be referred to as neurons. For example, the motion and/or image analysis functionality may include an input layer having a neuron for each pixel of an ultrasound image and/or voxel of an ultrasound volume. The output layer may have a neuron corresponding to each heart muscle, heart chamber, and/or any suitable anatomical structure. Each neuron of each layer may perform a processing function and pass the processed ultrasound image and/or motion information to one of a plurality of neurons of a downstream layer for further processing. As an example, neurons of a first layer may learn to recognize movement to obtain ultrasound images and/or volumes and to convert the movement into motion parameters. The neurons of a second layer may learn to position a series of ultrasound images based on the motion parameters from the first layer. The neurons of a third layer may learn to construct volumes from the obtained ultrasound images and/or volumes. The processing performed by the deep neural network may identify ultrasound probe motion parameters, including displacements along one or more planes and/or rotations, obtain ultrasound images, and construct ultrasound volumes with a high degree of probability.

In various embodiments, the positioning processor 160 may comprise suitable logic, circuitry, interfaces, and/or code that may be operable to generate a 3D ultrasound volume using displacement and/or rotation information obtained from the displacement processor 150 and cause a display system 134 to present the generated 3D ultrasound volume. For example, the positioning processor 150 may cause the display system to present a 3D ultrasound volume on the display 134. In some embodiments, an ultrasound operator may provide an input via the user input device 130 and/or touchscreen display 130, 134 to display a target view of the 3D ultrasound volume and/or to manipulate the 3D ultrasound volume. For example, the ultrasound operator may rotate the 3D ultrasound volume, zoom in on certain portions of the 3D ultrasound volume, zoom out, etc. Additionally and/or alternatively, the generated 3D ultrasound volume may be stored at archive 138 and/or any suitable computer readable medium.

Referring again to FIG. 1, the display system 134 may be any device capable of communicating visual information to a user. For example, a display system 134 may include a liquid crystal display, a light emitting diode display, and/or any suitable display or displays. The display system 134 can be operable to present 2D/3D ultrasound images 350, 360, 450, 460, 650, 660, 750, 760, 850, 860, and/or any suitable information.

The archive 138 may be one or more computer-readable memories integrated with the ultrasound system 100 and/or communicatively coupled (e.g., over a network) to the ultrasound system 100, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things.

In various embodiments, the archive 138 stores ultrasound images 350, 360, 450, 460, 650, 660, 750, 760, 850, 860, rendered 3D/4D volumes, instructions for acquiring ultrasound images 350, 360, 450, 460, 520, 540, 550, 560, 650, 660, 750, 760, 850, 860, instructions for obtaining first and second plane representations and/or first and second plane volume representations 330, 332, 340, 342, 430, 432, 440, 442, 630, 632, 640, 642, 644, 730, 732, 740, 742, 830, 832, 840, 842, 870, 872, 930, 940, 970, instructions to determine a center line 312, 412, 612, 712, 812, instructions to calculate displacements, rotations, and/or probe motion parameters 313, 413, 613, 615, 619, 621, 713, 715, 813, 814, 815, 816, 819, 1040, instructions for automatically detecting displacements, rotations, and/or probe motion parameters 313, 413, 613, 615, 619, 621, 713, 715, 813, 814, 815, 816, 819, 1040 in ultrasound images 350, 360, 450, 460, 650, 660, 750, 760, 850, 860, instructions for positioning ultrasound images sequentially using displacements, rotations, and/or probe motion parameters 313, 413, 613, 615, 619, 621, 713, 715, 813, 814, 815, 816, 819, 1040, and instructions for causing a display system 134 to present ultrasound images 350, 360, 450, 460, 650, 660, 750, 760, 850, 860 and/or generated ultrasound volumes.

Components of the ultrasound system 100 may be implemented in software, hardware, firmware, and/or the like. The various components of the ultrasound system 100 may be communicatively linked. Components of the ultrasound system 100 may be implemented separately and/or integrated in various forms. For example, the display system 134 and the user input device 130 may be integrated as a touchscreen display.

Still referring to FIG. 1, the training system 200 may comprise a training engine 210 and a training database 220. The training engine 210 may comprise suitable logic, circuitry, interfaces and/or code that may be operable to train the neurons of the deep neural network(s) (e.g., artificial intelligence model(s)) inferenced (i.e., deployed) by the image acquisition processor 140, the displacement processor 150, and/or the positioning processor 160. For example, the artificial intelligence model inferenced by the displacement processor 150 may be trained to automatically identify motion parameters from a bi-plane ultrasound image and/or volume using database(s) 220 of classified ultrasound images of anatomical structures. As another example, the artificial intelligence model inferenced by the displacement processor 150 and/or positioning processor 160 may be trained to automatically identify motion parameters, displacements, rotations, and the like in an ultrasound image using database(s) 220 of classified ultrasound images and/or motion parameters.

In various embodiments, the databases 220 of training images may be a Picture Archiving and Communication System (PACS), or any suitable data storage medium. In certain embodiments, the training engine 210 and/or training image databases 220 may be remote system(s) communicatively coupled via a wired or wireless connection to the ultrasound system 100 as shown in FIG. 1. Additionally and/or alternatively, components or all of the training system 200 may be integrated with the ultrasound system 100 in various forms. In some examples, the training image databases 220 may be integrated with the archive 138 or vice versa.

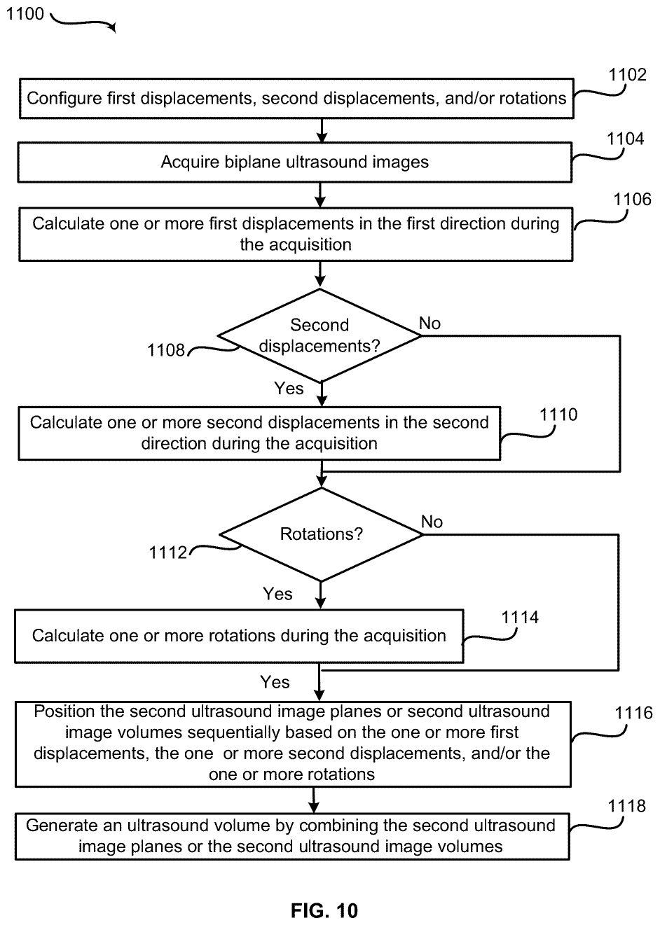

FIG. 10 is a flow chart 1100 illustrating exemplary steps 1102-1116 that may be utilized for obtaining an ultrasound volume from bi-plane ultrasound images, in accordance with various embodiments. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 1102, a signal processor 132, 140 of the ultrasound system 100 may be configured to acquire first displacements 313, 413, 613, 619, 713, 813, second displacements 615, 621, 715, and/or rotations 813, 814, 815, 816, 819 of biplane ultrasound images first ultrasound image planes 330, 332, 340, 342, 350, 360, 430, 432, 440, 442, 450, 460, 520, 540, 550, 560, 630, 632, 634, 640, 642, 644, 650, 660, 730, 732, 740, 742, 750, 760, 830, 832, 840, 842, 850, 860, 930, 940, 970. For example, an image acquisition processor may be configured to acquire a series of ultrasound images 330, 332, 340, 342, 350, 360, 430, 432, 440, 442, 450, 460, 520, 540, 550, 560, 630, 632, 634, 640, 642, 644, 650, 660, 730, 732, 740, 742, 750, 760, 830, 832, 840, 842, 850, 860, 930, 940, 970 using an ultrasound probe 104 that is moved across an anatomical structure. The acquired biplane ultrasound images 330, 332, 340, 342, 350, 360, 430, 432, 440, 442, 450, 460, 520, 540, 550, 560, 630, 632, 634, 640, 642, 644, 650, 660, 730, 732, 740, 742, 750, 760, 830, 832, 840, 842, 850, 860, 930, 940, 970 may be provided to the displacement processor 150 and/or stored at archive 138 and/or any suitable computer readable medium.

At step 1104, a signal processor 132, 140 of the ultrasound system 100 may be configured to acquire biplane ultrasound images 330, 332, 340, 342, 350, 360, 430, 432, 440, 442, 450, 460, 520, 540, 550, 560. For example, an image acquisition processor may be configured to acquire a series of ultrasound images 330, 332, 340, 342, 350, 360, 430, 432, 440, 442, 450, 460, 520, 540, 550, 560 using an ultrasound probe 104 that is moved across an anatomical structure. The acquired biplane ultrasound images 330, 332, 340, 342, 350, 360, 430, 432, 440, 442, 450, 460 may be provided to the displacement processor 150 and/or stored at archive 138 and/or any suitable computer readable medium.

At step 1106, a signal processor 132, 150 of the ultrasound system 100 may be configured to calculate one or more first displacements 313, 413, 613, 619, 713, 813 in the first direction during the acquisition of the biplane ultrasound images 330, 332, 340, 342, 350, 360, 430, 432, 440, 442, 450, 460, 520, 540, 550, 560. For example, a displacement processor 150 may be configured to calculate displacements 313, 413 in a first direction in the ultrasound images 330, 332, 340, 342, 350, 360, 430, 432, 440, 442, 450, 460, 520, 540, 550, 560. The displacements 313, 413 in the first direction may be stored by the displacement processor 150 in an archive 138 or other suitable computer readable medium, and/or provided to the positioning processor 160.

At step 1108, a signal processor 132, 150 of the ultrasound system 100 may be configured to determine whether to calculate second displacements 615, 621, 715 in a second direction. In some embodiments, the displacement processor 150 is configured with instructions indicating whether second displacements 615, 621, 715 should be calculated. Additionally and/or alternatively, an ultrasound operator may provide user input via the user input device 130 and/or touchscreen display 130, 134 indicating that second displacements 615, 621, 715 should be calculated. If no second displacements 615, 621, 715 are to be calculated, the signal processor 132 proceeds to step 1112.

At step 1110, a signal processor 132, 150 of the ultrasound system 100 may be configured to calculate one or more second displacements 615, 621, 715 in the second direction during the acquisition. For example, the displacement processor 150 may calculate one or more second displacements 615, 621, 715 in ultrasound images 630, 632, 634, 640, 642, 644, 650, 660, 730, 732, 740, 742, 750, 760. The second displacements 615, 621, 715 in the second direction may be stored by the displacement processor 150 in an archive 138 or other suitable computer readable medium, and/or provided to the positioning processor 160.

At step 1112, a signal processor 132, 150 of the ultrasound system 100 may be configured to determine whether to calculate rotations 813, 814, 815, 816, 819. In some embodiments, the displacement processor 150 is configured with instructions indicating whether the rotations 814, 815, 816, 819 should be calculated. Additionally and/or alternatively, an ultrasound operator may provide user input via the user input device 130 and/or touchscreen display 130, 134 indicating that the rotations 813, 814, 815, 816, 819 should be calculated. If no rotations 813, 814, 815, 816, 819 are to be calculated, the signal processor 132 proceeds to step 1116.

At step 1114, a signal processor 132, 150 of the ultrasound system 100 may be configured to calculate one or more rotations 813, 814, 815, 816, 819 during the acquisition. For example, the displacement processor 150 may calculate one or more rotations 813, 814, 815, 816, 819 in ultrasound images 830, 832, 840, 842, 850, 860, 870, 872. The rotations 813, 814, 815, 816, 819 may be stored by the displacement processor 150 in an archive 138 or other suitable computer readable medium, and/or provided to the positioning processor 160.

At step 1116, the signal processor 132, 160 of the ultrasound system 100 may be configured to position 510 second ultrasound image planes 340, 342, 520, 540, 550, 560, 640, 642, 644, 840, 842, or second ultrasound image volumes 440, 442, 520, 540, 550, 560, 740, 742, 870, 872, 940 sequentially based on the one or more first displacements 313, 413, 613, 619, 713, 813, the one or more second displacements 615, 621, 715, and/or the one or more rotations 813, 814, 815, 816, 819.

At step 1118, the signal processor 132, 160 of the ultrasound system 100 may be configured to generate an ultrasound volume 520 by combining the second ultrasound image planes 340, 342, 520, 540, 550, 560, 640, 642, 644, 840, 842 or the second ultrasound image volumes 440, 442, 520, 540, 550, 560, 740, 742, 870, 872, 940.

Aspects of the present disclosure provide a method 1100 and system 100 for acquiring an ultrasound volume comprising acquiring, by at least one processor 132, 140, sequential bi-plane ultrasound images 330, 332, 340, 342, 350, 360, 430, 432, 440, 442, 450, 460, 520, 540, 550, 560, 630, 632, 634, 640, 642, 644, 650, 660, 730, 732, 740, 742, 750, 760, 830, 832, 840, 842, 850, 860, 930, 940, 970 in a first direction using an ultrasound probe 104 of an ultrasound system 100, the sequential bi-plane ultrasound images 330, 332, 340, 342, 350, 360, 430, 432, 440, 442, 450, 460, 520, 540, 550, 560, 630, 632, 634, 640, 642, 644, 650, 660, 730, 732, 740, 742, 750, 760, 830, 832, 840, 842, 850, 860, 930, 940, 970 comprising first ultrasound image planes 330, 332, 430, 432, 630, 632, 634, 730, 732, 830, 832, 930 or first ultrasound image volumes 330, 332, 430, 432, 630, 632, 634, 730, 732, 830, 832, 930, 970 along a first plane and second ultrasound image planes 340, 342, 520, 540, 550, 560, 640, 642, 644, 840, 842 or second ultrasound image volumes 440, 442, 520, 540, 550, 560, 740, 742, 870, 872, 940 along a second plane, the first plane corresponding to the first direction and the second plane corresponding to a second direction.

The method 1100 may comprise, calculating, by the at least one processor 132, 150, one or more first displacements 313, 413, 613, 619, 713, 813 in the first direction of the sequential bi-plane ultrasound images 330, 332, 340, 342, 350, 360, 430, 432, 440, 442, 450, 460, 520, 540, 550, 560, 630, 632, 634, 640, 642, 644, 650, 660, 730, 732, 740, 742, 750, 760, 830, 832, 840, 842, 850, 860, 930, 940, 970 during the acquisition. The method 1100 may also comprise positioning 510, by the at least one processor 132, 160, the second ultrasound image planes 340, 342, 520, 540, 550, 560, 640, 642, 644, 840, 842 or the second ultrasound image volumes 440, 442, 520, 540, 550, 560, 740, 742, 870, 872, 940 sequentially, wherein the second ultrasound image planes 340, 342, 520, 540, 550, 560, 640, 642, 644, 840, 842 or the second ultrasound image volumes 440, 442, 520, 540, 550, 560, 740, 742, 870, 872, 940 are positioned 510 according to the one or more first displacements 313, 413, 613, 619, 713, 813. The method 1100 comprises generating, by the at least one processor 132, 160, the ultrasound volume by combining 520 the second ultrasound image planes 340, 342, 520, 540, 550, 560, 640, 642, 644, 840, 842 or the second ultrasound image volumes 440, 442, 520, 540, 550, 560, 740, 742, 870, 872, 940.

In an exemplary embodiment, the method 1100 comprises calculating, by the at least one processor 132, 150, one or more second displacements 615, 621, 715 along the second direction during the acquisition, wherein the second direction is at an angle to the first direction, and wherein the second ultrasound image planes 340, 342, 520, 540, 550, 560, 640, 642, 644, 840, 842 or the second ultrasound image volumes 440, 442, 520, 540, 550, 560, 740, 742, 870, 872, 940 are positioned 510, by the at least one processor 132, 160, according to the one or more first displacements 313, 413, 613, 619, 713, 813 and the one or more second displacements 615, 621, 715.

In an exemplary embodiment, the method 1100 comprises calculating, by the at least one processor 132, 150, one or more rotations 814, 815, 816, 819 during the acquisition, and wherein the second ultrasound image planes 340, 342, 520, 540, 550, 560, 640, 642, 644, 840, 842 or the second ultrasound image volumes 440, 442, 520, 540, 550, 560, 740, 742, 870, 872, 940 are positioned, by the at least one processor 132, 160, according to the one or more first displacements 313, 413, 613, 619, 713, 813, the one or more second displacements 615, 621, 715, and the one or more rotations 813, 814, 815, 816, 819.

In an exemplary embodiment, the method 1100 comprises calculating, by the at least one processor 132, 150, one or more third displacements of one or more additional planes during the acquisition, and wherein the second ultrasound image planes 340, 342, 520, 540, 550, 560, 640, 642, 644, 840, 842 or the second ultrasound image volumes 440, 442, 520, 540, 550, 560, 740, 742, 870, 872, 940 are positioned, by the at least one processor 132, 160, according to the one or more first displacements 313, 413, 613, 619, 713, 813, the one or more second displacements 615, 621, 715, and the one or more rotations 813, 814, 815, 816, 819.

In an exemplary embodiment, the method 1100 comprises generating the ultrasound volume comprises combining 510 a subset of the second ultrasound image planes 340, 342, 520, 540, 550, 560, 640, 642, 644, 840, 842 or the second ultrasound image volumes 440, 442, 520, 540, 550, 560, 740, 742, 870, 872, 940. In an exemplary embodiment, the method 1100 comprises obtaining the one or more first displacements 313, 413, 613, 619, 713, 813 via an artificial intelligence model 1000.

In an exemplary embodiment, the calculating of the one or more first displacements 313, 413, 613, 619, 713, 813, by the at least one processor 132, 150, is based on a normalized cross-correlation between each of the sequential bi-plane ultrasound images 330, 332, 340, 342, 350, 360, 430, 432, 440, 442, 450, 460, 520, 540, 550, 560, 630, 632, 634, 640, 642, 644, 650, 660, 730, 732, 740, 742, 750, 760, 830, 832, 840, 842, 850, 860, 930, 940, 970 or sequential bi-plane ultrasound image volumes 330, 332, 340, 342, 350, 360, 430, 432, 440, 442, 450, 460, 520, 540, 550, 560, 630, 632, 634, 640, 642, 644, 650, 660, 730, 732, 740, 742, 750, 760, 830, 832, 840, 842, 850, 860, 930, 940, 970.

Various embodiments provide an ultrasound system 100 for acquiring an ultrasound volume 570 comprising an ultrasound probe 104 configured to acquire sequential bi-plane ultrasound images 330, 332, 340, 342, 350, 360, 430, 432, 440, 442, 450, 460, 520, 540, 550, 560, 630, 632, 634, 640, 642, 644, 650, 660, 730, 732, 740, 742, 750, 760, 830, 832, 840, 842, 850, 860, 930, 940, 970, and at least one processor configured to acquire sequential bi-plane ultrasound images 330, 332, 340, 342, 350, 360, 430, 432, 440, 442, 450, 460, 520, 540, 550, 560, 630, 632, 634, 640, 642, 644, 650, 660, 730, 732, 740, 742, 750, 760, 830, 832, 840, 842, 850, 860, 930, 940, 970 in a first direction using the ultrasound probe 104, the sequential bi-plane ultrasound images 330, 332, 340, 342, 350, 360, 430, 432, 440, 442, 450, 460, 520, 540, 550, 560, 630, 632, 634, 640, 642, 644, 650, 660, 730, 732, 740, 742, 750, 760, 830, 832, 840, 842, 850, 860, 930, 940, 970 comprising first ultrasound image planes 330, 332, 430, 432, 630, 632, 634, 730, 732, 830, 832, 930 or first ultrasound image volumes 330, 332, 430, 432, 630, 632, 634, 730, 732, 830, 832, 930, 970 along a first plane and second ultrasound image planes 340, 342, 520, 540, 550, 560, 640, 642, 644, 840, 842 or second ultrasound image volumes 440, 442, 520, 540, 550, 560, 740, 742, 870, 872, 940 along a second plane, the first plane corresponding to the first direction and the second plane corresponding to a second direction.

In a representative embodiment, the at least one processor 132, 150 may be configured to calculate one or more first displacements 313, 413, 613, 619, 713, 813 in the first direction of the sequential bi-plane ultrasound images 330, 332, 340, 342, 350, 360, 430, 432, 440, 442, 450, 460, 520, 540, 550, 560, 630, 632, 634, 640, 642, 644, 650, 660, 730, 732, 740, 742, 750, 760, 830, 832, 840, 842, 850, 860, 930, 940, 970 during the acquisition, position 510 the second ultrasound image planes 340, 342, 520, 540, 550, 560, 640, 642, 644, 840, 842 or the second ultrasound image volumes 440, 442, 520, 540, 550, 560, 740, 742, 870, 872, 940 sequentially, wherein the second ultrasound image planes 340, 342, 520, 540, 550, 560, 640, 642, 644, 840, 842 or the second ultrasound image volumes 440, 442, 520, 540, 550, 560, 740, 742, 870, 872, 940 are positioned according to the one or more first displacements 313, 413, 613, 619, 713, 813, and generate the ultrasound volume 570 by combining 510 the second ultrasound image planes 340, 342, 520, 540, 550, 560, 640, 642, 644, 840, 842 or the second ultrasound image volumes 440, 442, 520, 540, 550, 560, 740, 742, 870, 872, 940.

In a representative embodiment, the at least one processor 132, 150 may be configured to calculate one or more second displacements 615, 621, 715 along the second direction during the acquisition, wherein the second direction is at an angle to the first direction, and wherein the second ultrasound image planes 340, 342, 520, 540, 550, 560, 640, 642, 644, 840, 842 or the second ultrasound image volumes 440, 442, 520, 540, 550, 560, 740, 742, 870, 872, 940 are positioned according to the one or more first displacements 313, 413, 613, 619, 713, 813 and the one or more second displacements 615, 621, 715. In a representative embodiment, the at least one processor 132, 150 may be configured to calculate one or more rotations 813, 814, 815, 816, 819 during the acquisition, and wherein the second ultrasound image planes 340, 342, 520, 540, 550, 560, 640, 642, 644, 840, 842 or the second ultrasound image volumes 440, 442, 520, 540, 550, 560, 740, 742, 870, 872, 940 are positioned according to the one or more first displacements 313, 413, 613, 619, 713, 813, the one or more second displacements 615, 621, 715, and the one or more rotations 813, 814, 815, 816, 819.

In a representative embodiment, the at least one processor 132, 150 may be configured to calculate one or more third displacements of one or more additional planes during the acquisition, and wherein the second ultrasound image planes 340, 342, 520, 540, 550, 560, 640, 642, 644, 840, 842 or the second ultrasound image volumes 440, 442, 520, 540, 550, 560, 740, 742, 870, 872, 940 are positioned, by the at least one processor 132, 160, according to the one or more first displacements 313, 413, 613, 619, 713, 813, the one or more second displacements 615, 621, 715, and the one or more third displacements. In a representative embodiment, the at least one processor 132, 160 may be configured to generate the ultrasound volume 570 by combining a subset 522 of the second ultrasound image planes 340, 342, 520, 540, 550, 560, 640, 642, 644, 840, 842 or the second ultrasound image volumes 440, 442, 520, 540, 550, 560, 740, 742, 870, 872, 940.

In a representative embodiment, the at least one processor 132, 150 may be configured to calculate the one or more first displacements 313, 413, 613, 619, 713, 813 based on a normalized cross-correlation between each of the sequential bi-plane ultrasound images 330, 332, 340, 342, 350, 360, 430, 432, 440, 442, 450, 460, 520, 540, 550, 560, 630, 632, 634, 640, 642, 644, 650, 660, 730, 732, 740, 742, 750, 760, 830, 832, 840, 842, 850, 860, 930, 940, 970 or sequential bi-plane ultrasound image volumes 330, 332, 340, 342, 350, 360, 430, 432, 440, 442, 450, 460, 520, 540, 550, 560, 630, 632, 634, 640, 642, 644, 650, 660, 730, 732, 740, 742, 750, 760, 830, 832, 840, 842, 850, 860, 930, 940, 970.

Various embodiments provide an ultrasound system 100 for acquiring an ultrasound volume 570 comprising an ultrasound probe 104 configured to acquire sequential bi-plane ultrasound images 330, 332, 340, 342, 350, 360, 430, 432, 440, 442, 450, 460, 520, 540, 550, 560, 630, 632, 634, 640, 642, 644, 650, 660, 730, 732, 740, 742, 750, 760, 830, 832, 840, 842, 850, 860, 930, 940, 970 and at least one processor 132, 140 configured to acquire sequential bi-plane ultrasound images 330, 332, 340, 342, 350, 360, 430, 432, 440, 442, 450, 460, 520, 540, 550, 560, 630, 632, 634, 640, 642, 644, 650, 660, 730, 732, 740, 742, 750, 760, 830, 832, 840, 842, 850, 860, 930, 940, 970 in a first direction using the ultrasound probe 104, the sequential bi-plane ultrasound images 330, 332, 340, 342, 350, 360, 430, 432, 440, 442, 450, 460, 520, 540, 550, 560, 630, 632, 634, 640, 642, 644, 650, 660, 730, 732, 740, 742, 750, 760, 830, 832, 840, 842, 850, 860, 930, 940, 970 comprising first ultrasound image planes 330, 332, 430, 432, 630, 632, 634, 730, 732, 830, 832, 930 or first ultrasound image volumes 330, 332, 430, 432, 630, 632, 634, 730, 732, 830, 832, 930, 970 along a first plane and second ultrasound image planes 340, 342, 520, 540, 550, 560, 640, 642, 644, 840, 842 or second ultrasound image volumes 440, 442, 520, 540, 550, 560, 740, 742, 870, 872, 940 along a second plane, the first plane corresponding to the first direction and the second plane corresponding to a second direction.

In a representative embodiment, the at least one processor 132, 150 may be configured to calculate one or more first displacements 313, 413, 613, 619, 713, 813 in the first direction of the sequential bi-plane ultrasound images 330, 332, 340, 342, 350, 360, 430, 432, 440, 442, 450, 460, 520, 540, 550, 560, 630, 632, 634, 640, 642, 644, 650, 660, 730, 732, 740, 742, 750, 760, 830, 832, 840, 842, 850, 860, 930, 940, 970 and calculate one or more second displacements 615, 621, 715 along the second direction during the acquisition, wherein the second direction is at an angle to the first direction, position the second ultrasound image planes or the second ultrasound image volumes sequentially, wherein the second ultrasound image planes 340, 342, 520, 540, 550, 560, 640, 642, 644, 840, 842 or the second ultrasound image volumes 440, 442, 520, 540, 550, 560, 740, 742, 870, 872, 940 are positioned according to the one or more first displacements 313, 413, 613, 619, 713, 813 and the one or more second displacements 615, 621, 715. In a representative embodiment, the at least one processor 132, 160 may be configured to generate the ultrasound volume 570 by combining 510 the second ultrasound image planes 340, 342, 520, 540, 550, 560, 640, 642, 644, 840, 842 or the second ultrasound image volumes 440, 442, 520, 540, 550, 560, 740, 742, 870, 872, 940.

In a representative embodiment, the at least one processor 132, 150 may be configured to calculate one or more rotations 813, 814, 815, 816, 819 during the acquisition, and wherein the second ultrasound image planes 340, 342, 520, 540, 550, 560, 640, 642, 644, 840, 842 or the second ultrasound image volumes 440, 442, 520, 540, 550, 560, 740, 742, 870, 872, 940 are positioned according to the one or more first displacements 313, 413, 613, 619, 713, 813, the one or more second displacements, and the one or more rotations 813, 814, 815, 816, 819. In a representative embodiment, the at least one processor 132, 150 may be configured to calculate one or more third displacements of one or more additional planes during the acquisition, and wherein the second ultrasound image planes 340, 342, 520, 540, 550, 560, 640, 642, 644, 840, 842 or the second ultrasound image volumes 440, 442, 520, 540, 550, 560, 740, 742, 870, 872, 940 are positioned 510, by the at least one processor 132, 160, according to the one or more first displacements 313, 413, 613, 619, 713, 813, the one or more second displacements 615, 621, 715, and the one or more third displacements.

In a representative embodiment, the at least one processor 132, 160 may be configured to generate the ultrasound volume 570 by combining a subset 522 of the second ultrasound image planes 340, 342, 520, 540, 550, 560, 640, 642, 644, 840, 842 or the second ultrasound image volumes 440, 442, 520, 540, 550, 560, 740, 742, 870, 872, 940. In a representative embodiment, the at least one processor 132, 150 may be configured to calculate the one or more first displacements 313, 413, 613, 619, 713, 813 based on a normalized cross-correlation between each of the sequential bi-plane ultrasound image planes 330, 332, 340, 342, 350, 360, 430, 432, 440, 442, 450, 460, 520, 540, 550, 560, 630, 632, 634, 640, 642, 644, 650, 660, 730, 732, 740, 742, 750, 760, 830, 832, 840, 842, 850, 860, 930, 940, 970 or sequential bi-plane ultrasound image volumes 330, 332, 340, 342, 350, 360, 430, 432, 440, 442, 450, 460, 520, 540, 550, 560, 630, 632, 634, 640, 642, 644, 650, 660, 730, 732, 740, 742, 750, 760, 830, 832, 840, 842, 850, 860, 930, 940, 970.

In a representative embodiment, the at least one processor 132, 150 may be configured to calculate the one or more first displacements 313, 413, 613, 619, 713, 813 via an artificial intelligence model 1000. In a representative embodiment, the at least one processor 132, 160 may be configured to generate a three dimensional volume 570.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” and/or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for acquiring a target ultrasound image having a target view of one or more anatomical structures.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A method for acquiring an ultrasound volume comprising:
acquiring, by at least one processor, sequential bi-plane ultrasound images in a first direction using an ultrasound probe of an ultrasound system, the sequential bi-plane ultrasound images comprising first ultrasound image planes or first ultrasound image volumes along a first plane and second ultrasound image planes or second ultrasound image volumes along a second plane, the first plane corresponding to the first direction and the second plane corresponding to a second direction;
calculating, by the at least one processor, one or more first displacements in the first direction of the sequential bi-plane ultrasound images during the acquisition;
positioning, by the at least one processor, the second ultrasound image planes or the second ultrasound image volumes sequentially, wherein the second ultrasound image planes or the second ultrasound image volumes are positioned according to the one or more first displacements; and
generating the ultrasound volume, by the at least one processor, by combining the second ultrasound image planes or the second ultrasound image volumes.

2. The method of claim 1, further comprising calculating, by the at least one processor, one or more second displacements along the second direction during the acquisition, wherein the second direction is at an angle to the first direction, and wherein the second ultrasound image planes or the second ultrasound image volumes are positioned, by the at least one processor, according to the one or more first displacements and the one or more second displacements.

3. The method of claim 2, further comprising calculating, by the at least one processor, one or more rotations during the acquisition, and wherein the second ultrasound image planes or the second ultrasound image volumes are positioned, by the at least one processor, according to the one or more first displacements, the one or more second displacements, and the one or more rotations.

4. The method of claim 2, further comprising calculating, by the at least one processor, one or more third displacements of one or more additional planes during the acquisition, and wherein the second ultrasound image planes or the second ultrasound image volumes are positioned, by the at least one processor, according to the one or more first displacements, the one or more second displacements, and the one or more third displacements.

5. The method of claim 1, wherein the generating the ultrasound volume comprises combining a subset of the second ultrasound image planes or the second ultrasound image volumes.

6. The method of claim 1, wherein the one or more first displacements are obtained via an artificial intelligence model.

7. The method of claim 1, wherein the calculating of the one or more first displacements is based on a normalized cross-correlation between each of the sequential bi-plane ultrasound images or sequential bi-plane ultrasound image volumes.

8. An ultrasound system for acquiring an ultrasound volume comprising:
an ultrasound probe configured to acquire sequential bi-plane ultrasound images;
at least one processor configured to:
acquire the sequential bi-plane ultrasound images in a first direction using the ultrasound probe, the sequential bi-plane ultrasound images comprising first ultrasound image planes or first ultrasound image volumes along a first plane and second ultrasound image planes or second ultrasound image volumes along a second plane, the first plane corresponding to the first direction and the second plane corresponding to a second direction;
calculate one or more first displacements in the first direction of the sequential bi-plane ultrasound images during the acquisition;
position the second ultrasound image planes or the second ultrasound image volumes sequentially, wherein the second ultrasound image planes or the second ultrasound image volumes are positioned according to the one or more first displacements; and
generate the ultrasound volume by combining the second ultrasound image planes or the second ultrasound image volumes.

9. The ultrasound system of claim 8, wherein the at least one processor is further configured to calculate one or more second displacements along the second direction during the acquisition, wherein the second direction is at an angle to the first direction, and wherein the second ultrasound image planes or the second ultrasound image volumes are positioned according to the one or more first displacements and the one or more second displacements.

10. The ultrasound system of claim 9, wherein the at least one processor is further configured to calculate one or more rotations during the acquisition, and wherein the second ultrasound image planes or the second ultrasound image volumes are positioned according to the one or more first displacements, the one or more second displacements, and the one or more rotations.

11. The ultrasound system of claim 9, wherein the at least one processor is further configured to calculate one or more third displacements of one or more additional planes during the acquisition, and wherein the second ultrasound image planes or the second ultrasound image volumes are positioned according to the one or more first displacements, the one or more second displacements, and the one or more third displacements.

12. The ultrasound system of claim 9, wherein the at least one processor is configured to generate the ultrasound volume by combining a subset of the second ultrasound image planes or the second ultrasound image volumes.

13. The ultrasound system of claim 8, wherein the at least one processor is configured to calculate the one or more first displacements is based on a normalized cross-correlation between each of the sequential bi-plane ultrasound images or sequential bi-plane ultrasound image volumes.

14. An ultrasound system for acquiring an ultrasound volume comprising:
an ultrasound probe configured to acquire sequential bi-plane ultrasound images;
at least one processor configured to:
acquire the sequential bi-plane ultrasound images in a first direction using the ultrasound probe, the sequential bi-plane ultrasound images comprising first ultrasound image planes or first ultrasound image volumes along a first plane and second ultrasound image planes or second ultrasound image volumes along a second plane, the first plane corresponding to the first direction and the second plane corresponding to a second direction;
calculate one or more first displacements in the first direction of the sequential bi-plane ultrasound images and calculate one or more second displacements along the second direction during the acquisition, wherein the second direction is at an angle to the first direction;
position the second ultrasound image planes or the second ultrasound image volumes sequentially, wherein the second ultrasound image planes or the second ultrasound image volumes are positioned according to the one or more first displacements and the one or more second displacements; and
generate the ultrasound volume by combining the second ultrasound image planes or the second ultrasound image volumes.

15. The ultrasound system of claim 14, wherein the at least one processor is further configured to calculate one or more rotations during the acquisition, and wherein the second ultrasound image planes or the second ultrasound image volumes are positioned according to the one or more first displacements, the one or more second displacements, and the one or more rotations.

16. The ultrasound system of claim 14, wherein the at least one processor is further configured to calculate one or more third displacements of one or more additional planes during the acquisition, and wherein the second ultrasound image planes or the second ultrasound image volumes are positioned according to the one or more first displacements, the one or more second displacements, and the one or more third displacements.

17. The ultrasound system of claim 14, wherein the at least one processor is configured to generate the ultrasound volume by combining a subset of the second ultrasound image planes or the second ultrasound image volumes.

18. The ultrasound system of claim 14, wherein the at least one processor is configured to calculate the one or more first displacements based on a normalized cross-correlation between each of the sequential bi-plane ultrasound images or sequential bi-plane ultrasound image volumes.

19. The ultrasound system of claim 14, wherein the at least one processor is configured to calculate the one or more first displacements are obtained via an artificial intelligence model.

20. The ultrasound system of claim 14, wherein the ultrasound volume generated by the at least one processor is a three dimensional volume.
